(19) United States
(12) Patent Application Publication
(10) Pub. No.: US 2018/0170825 A1
(43) Pub. Date: Jun. 21, 2018

(54) PROCESS AND SYSTEM FOR LOW PRESSURE OLEFIN CONVERSION TO A DISTILLATE BOILING RANGE PRODUCT

(71) Applicant: ExxonMobil Research and Engineering Company, Annandale, NJ (US)

(72) Inventors: Samia ILIAS, Bridgewater, NJ (US); Brett T. Loveless, Houston, TX (US); Stephen J. McCarthy, Center Valley, PA (US); Brandon J. O'NEILL, Lebanon, NJ (US)

(21) Appl. No.: 15/830,171

(22) Filed: Dec. 4, 2017

Related U.S. Application Data
(60) Provisional application No. 62/437,118, filed on Dec. 21, 2016.

(51) Int. Cl. C07C 2/12; C07C 1/20; B01J 29/40; B01J 29/70

(52) U.S. Cl. CPC C07C 2/12; C07C 1/20; B01J 29/40; B01J 29/7046; B01J 29/703; C07C 2529/70; B01J 29/7026; B01J 29/7038; B01J 29/70; C07C 2529/40; B01J 29/7034

(57) ABSTRACT

Processes and reaction systems for low pressure oligomerization of olefins to produce distillate boiling range products using zeolite catalysts are provided herein.

### CROSS-REFERENCE TO RELATED APPLICATIONS

This application claims the benefit of U.S. Provisional Application No. 62/437,118, filed on Dec. 21, 2016, the entire contents of which are incorporated herein by reference.

### FIELD

This invention relates to processes and systems for low pressure oligomerization of olefins to produce distillate boiling range products.

### BACKGROUND

Demand for diesel fuel and other distillate products (e.g., heating oil, kerosene, jet fuel) is believed to outpace current supply and may even exceed demand for gasoline. Thus, there is a need for further techniques for producing such distillate products.

Light olefins are produced in typical hydrocarbon refining operations that also produce gasoline and distillate products. Additionally, it is possible to obtain olefins from natural gas and coal sources via conversion of methanol and other oxygenates via the use of zeolite catalysts. To supplement diesel production from newly-extracted crude oil and to meet the rising demand for diesel and other distillates, processes were developed to convert olefins to yield additional diesel and other distillate products as well as gasoline products. For example, Mobil developed the Mobil Olefins to Gasoline and Distillate (MOGD) process where an H-ZSM-5 based catalyst may be used to convert C₃-C₄ olefins to gasoline and distillate products.

However, current commercial processes for olefin oligomerization typically require higher pressure, preferably pressures higher than 300 psig, and more preferably pressures higher than 600 psig, in order to obtain high olefin conversion. Operating at such high pressures can result in increased energy costs. Thus, there remains a need for providing more economically efficient methods of converting olefins to diesel and other distillate products at lower pressures while still maintaining high yields of distillate product.

### SUMMARY

It has been found that high conversion of olefins to distillate boiling range products via oligomerization may be achieved under advantageously lower pressure (e.g., less than 200 psig) by using 10-membered ring or 12-membered ring zeolite catalysts, particularly zeolite catalysts comprising a zeolite having a framework structure of BEA, FER, MRE, MFI, MEL, MWW, MTT, MTW, MFS, MOR, MEI, ITN, TON and a combination thereof

Thus, in one aspect, embodiments of the invention provide a process for oligomerizing olefins to produce a distillate boiling range product. The process comprises contacting a feed consisting essentially of C₂-C₄ olefins with an oligomerization catalyst comprising a zeolite having a framework structure selected from the group consisting of BEA, FER, MRE, MFI, MEL, MWW, MTT, MTW, MFS, MOR, MEI, ITN, TON and a combination thereof in at least one reactor operating under suitable conditions to oligomerize at least a portion of the olefins to produce an effluent comprising the distillate boiling range product, wherein the at least one reactor operates at a pressure below 200 psig.

In still another aspect, embodiments of the invention provide another process for oligomerizing olefins to produce a distillate boiling range product. The process comprises contacting a feed comprising olefins with an oligomerization catalyst comprising a zeolite having a framework structure of MRE in at least one reactor operating under suitable conditions to oligomerize at least a portion of the olefins to produce an effluent comprising the distillate boiling range product, wherein the at least one reactor operates at a pressure below 200 psig.

In still another aspect, embodiments of the invention provide another process for oligomerizing olefins to produce a distillate boiling range product. The process comprises contacting a feed comprising olefins with an oligomerization catalyst in at least one reactor operating under suitable conditions to oligomerize at least a portion of the olefins to produce an effluent comprising the distillate boiling range product; wherein the at least one reactor operates at a pressure below 200 psig; and wherein the oligomerization catalyst comprises a zeolite having a framework structure selected from the group consisting of BEA, FER, MRE, MFI, MEL, MWW, MTT, MTW, MFS, MOR, MEI, ITN, TON and a combination thereof; and the oligomerization catalyst has the following: (i) a silicon to aluminum molar ratio of about 20 to about 100; (ii) a surface area greater than about 150 m²/g; and (iii) a hexane cracking activity of greater than about 20.

In still another aspect, embodiments of the invention provide a process for producing a distillate boiling range product. The process comprises: contacting a first stream comprising methanol and/or dimethyl ether with a methanol conversion catalyst under suitable conditions to produce an intermediate product comprising olefins; and contacting the intermediate product with an oligomerization catalyst comprising a zeolite having a framework structure selected from the group consisting of BEA, FER, MRE, MFI, MEL, MWW, MTT, MTW, MFS, MOR, MEI, ITN, TON and a combination thereof at a pressure below 200 psig to oligomerize at least a portion of the olefins to produce the distillate boiling range product.

In still another aspect, embodiments of the invention provide a reaction system for oligomerizing olefins to produce a distillate boiling range product. The system comprises: a feed stream consisting essentially of C₂-C₄ olefins; an effluent stream comprising the distillate boiling range product; and at least one reactor operated under suitable conditions to oligomerize at least a portion of the olefins to the distillate boiling range product, wherein the at least one reactor comprises: a feed stream inlet for providing the feed stream to the reaction system; a catalyst comprising a zeolite having a framework structure selected from the group consisting of BEA, FER, MRE, MFI, MEL, MWW, MTT, MTW, MFS, MOR, MEI, ITN, TON and a combination thereof; and an effluent outlet for removal of the effluent stream; and wherein the at least one reactor operates at a pressure below 200 psig.

Other embodiments, including particular aspects of the embodiments summarized above, will be evident from the detailed description that follows.

### BRIEF DESCRIPTION OF THE DRAWINGS

FIG. 1 illustrates effect of pressure on product yields from propene conversion using an H-ZSM-48 catalyst at 200° C. and a weight hourly space velocity (WHSV) of 1.67.

FIG. 2 illustrates effect of pressure on product yields from 1-pentene conversion using an H-ZSM-48 catalyst at 225° C. and a WHSV of 1.67.

FIG. 3 illustrates effect of zeolite framework (all in hydrogen form) on product yields from propene conversion at 200° C., 800 psig and a WHSV of 1.67.

FIG. 4 illustrates product yields and propene conversion using H-ZSM-5 and H-ZSM-48 catalysts at 200° C., 90 psig and a WHSV of 1.67.

FIG. 5 illustrates effect of pressure on product yields from propene conversion using MCM-49, ZSM-5 and ZSM-48 catalysts at 200° C. and a WHSV of 1.67.

### DETAILED DESCRIPTION

In various aspects of the invention, catalysts and methods for preparing catalysts are provided.

### I. Definitions

For purposes of this invention and the claims hereto, the numbering scheme for the Periodic Table Groups is according to the IUPAC Periodic Table of Elements.

The term “and/or” as used in a phrase such as “A and/or B” herein is intended to include “A and B”, “A or B”, “A”, and “B”.

The terms “substituent”, “radical”, “group”, and “moiety” may be used interchangeably.

As used herein, and unless otherwise specified, the term “Cₙ” means hydrocarbon(s) having n carbon atom(s) per molecule, wherein n is a positive integer.

As used herein, and unless otherwise specified, the term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n.

As used herein, the phrase “at least a portion of” means >0 to 100.0 wt % of the composition to which the phrase refers. The phrase “at least a portion of” refers to an amount ≤about 1.0 wt %, ≤about 2.0 wt %, ≤about 5.0 wt %, ≤about 10.0 wt %, ≤about 20.0 wt %, ≤about 25.0 wt %, ≤about 30.0 wt %, ≤about 40.0 wt %, ≤about 50.0 wt %, ≤about 60.0 wt %, ≤about 70.0 wt %, ≤about 75.0 wt %, ≤about 80.0 wt %, ≤about 90.0 wt %, ≤about 95.0 wt %, ≤about 98.0 wt %, ≤about 99.0 wt %, or ≤about 100.0 wt %. Additionally or alternatively, the phrase “at least a portion of” refers to an amount ≥about 1.0 wt %, ≥about 2.0 wt %, ≥about 5.0 wt %, ≥about 10.0 wt %, ≥about 20.0 wt %, ≥about 25.0 wt %, ≥about 30.0 wt %, ≥about 40.0 wt %, ≥about 50.0 wt %, ≥about 60.0 wt %, ≥about 70.0 wt %, ≥about 75.0 wt %, ≥about 80.0 wt %, ≥about 90.0 wt %, ≥about 95.0 wt %, ≥about 98.0 wt %, ≥about 99.0 wt %, or about 100.0 wt %. Ranges expressly disclosed include combinations of any of the above-enumerated values; e.g., about 10.0 to about 100.0 wt %, about 10.0 to about 98.0 wt %, about 2.0 to about 10.0 wt %, about 40.0 to 60.0 wt %, etc.

As used herein, and unless otherwise specified, the term “aromatic” refers to unsaturated cyclic hydrocarbons having a delocalized conjugated π system and having from 4 to 20 carbon atoms (aromatic C₄-C₂ hydrocarbon). Exemplary aromatics include, but are not limited to benzene, toluene, xylenes, mesitylene, ethylbenzenes, cumene, naphthalene, methylnaphthalene, dimethylnaphthalenes, ethylnaphthalenes, acenaphthalene, anthracene, phenanthrene, tetraphene, naphthacene, benzanthracenes, fluoranthrene, pyrene, chrysene, triphenylene, and the like, and combinations thereof. The aromatic may optionally be substituted, e.g., with one or more alkyl group, alkoxy group, halogen, etc. Additionally, the aromatic may comprise one or more heteroatoms. Examples of heteroatoms include, but are not limited to, nitrogen, oxygen, and/or sulfur. Aromatics with one or more heteroatom include, but are not limited to furan, benzofuran, thiophene, benzothiophene, oxazole, thiazole and the like, and combinations thereof. The aromatic may comprise monocyclic, bicyclic, tricyclic, and/or polycyclic rings (in some embodiments, at least monocyclic rings, only monocyclic and bicyclic rings, or only monocyclic rings) and may be fused rings.

As used herein, the term “olefin” refers to an unsaturated hydrocarbon chain of 2 to about 40 carbon atoms in length containing at least one carbon-to-carbon double bond, preferably 2 to 20 carbons in length, preferably 2 to 12 carbons in length, preferably 2 to 5 carbons in length, preferably 2 to 4 carbons in length, preferably ethylene, propylene, butene, pentene, hexene, heptene, octene, nonene, decene, undecene, dodecene, and isomers thereof. The olefin may be straight-chain, branched-chain or cyclic. “Olefin” is intended to embrace all structural isomeric forms of olefins. As used herein, the term “light olefin” refers to olefins having 2 to 5 carbon atoms (i.e., ethene, propene, butenes and pentenes).

As used herein, and unless otherwise specified, the term “paraffin,” alternatively referred to as “alkane,” refers to a saturated hydrocarbon chain of 1 to about 40 carbon atoms in length, such as, but not limited to methane, ethane, propane and butane. The paraffin may be straight-chain, cyclic or branched-chain. “Paraffin” is intended to embrace all structural isomeric forms of paraffins. The term “non-cyclic paraffin” refers to straight-chain or branched-chain paraffins. The term “isoparaffin” refer to branched-chain paraffin, and the term “n-paraffin” or “normal paraffin” refers to straight-chain paraffins.

As used herein, the term “gasoline” or “gasoline boiling range” refers to a composition containing at least predominantly C₅-C₁₂ hydrocarbons. In one embodiment, gasoline or gasoline boiling range components is further defined to refer to a composition containing at least predominantly C₅-C₁₂ hydrocarbons and further having a boiling range of from about 100° F. to up to 330° F. In an alternative embodiment, gasoline or gasoline boiling range components is defined to refer to a composition containing at least predominantly C₅-C₁₂ hydrocarbons, having a boiling range of from about 100° F. to up to 330° F., and further defined to meet ASTM standard D4814.

As used herein, and unless specified otherwise, the term “distillate” or “distillate boiling range” refers to a composition containing predominately C₁₀-C₂₅ hydrocarbons. In one embodiment, distillate or distillate boiling range products are further defined to refer to a composition containing at least predominately C₁₀-C₂₅ hydrocarbons and further having a boiling range of from about 330° F. to about 1100° F. In particular, distillate or distillate boiling range products may have a T₁₀ of at least about 330° F. and a T₉₀ of less than about 730° F. Examples of distillates or distillate boiling range products include, but are not limited to, naphtha, jet fuel, diesel, kerosene, aviation gas, fuel oil, and blends thereof.

As used herein, and unless specified otherwise, the term “diesel” refers to middle distillate fuels containing at least predominantly C₁₂-C₂₅ hydrocarbons. In one embodiment, diesel is further defined to refer to a composition containing at least predominantly C₁₂-C₂₅ hydrocarbons, and further having a boiling range of from about 330° F. to about 700° F. In an alternative embodiment, diesel is as defined above to refer to a composition containing at least predominantly C₁₂-C₂₅ hydrocarbons, having a boiling range of from about 330° F. to about 700° F., and further defined to meet ASTM standard D975.

As used herein, the term “naphtha” or “naphtha boiling range” refers to a middle boiling range hydrocarbon fraction or fractions, typically including between about three and twenty carbon atoms, which are major components of gasoline. In one embodiment, naphtha or naphtha boiling range components is further defined to have a boiling range distribution between about 38° C. and about 200° C. at 0.101 MPa, and further defined to meet ASTM standard D5307.

### II. Processes for Oligomerizing Olefins to Distillate Boiling Range Product

The invention relates to various processes for oligomerizing olefins to distillate boiling range products, particularly at lower pressures. The process may comprise contacting a feed comprising olefins with an oligomerization catalyst in at least one reactor operating under suitable conditions to oligomerize at least a portion of the olefins to form oligomerized olefins to produce an effluent comprising the distillate boiling range product.

### II.A. Olefin-Containing Feed

The feed described herein may comprise C₂-C₄₀ olefins, preferably C₂-C₂₀ olefins, preferably C₂-C₁₂ olefins, preferably C₂-C₅ olefins, preferably C₂-C₄ olefins. In one embodiment the feed may comprise, consist essentially of, or consist of C₂-C₅ olefins or C₂-C₄ olefins.

The feed may comprise olefins in an amount of at least about 5.0 wt %, at least about 10 wt %, at least about 20 wt %, at least about 30 wt %, at least about 40 wt %, at least about 50 wt %, at least about 60 wt %, at least about 65 wt %, at least about 70 wt %, at least about 75 wt %, at least about 80 wt %, at least about 85 wt %, at least about 90 wt %, at least about 95 wt %, at least about 97 wt %, at least about 99 wt % or up to about 100 wt %. Additionally or alternatively, the feed may comprise olefins in an amount of about 5.0 wt % to about 20 wt %, about 5.0 wt % to about 10 wt %, about 5.0 wt % to about 100 wt %, about 20 wt % to about 100 wt %, about 50 wt % to about 100 wt %, about 60 wt % to about 100 wt %, about 60 wt % to about 99 wt %, about 60 wt % to about 97 wt %, about 60 wt % to about 95 wt %, about 60 wt % to about 90 wt %, about 60 wt % to about 85 wt %, about 70 wt % to about 100 wt %, about 70 wt % to about 99 wt %, about 70 wt % to about 97 wt %, about 70 wt % to about 95 wt %, about 70 wt % to about 90 wt %, about 70 wt % to about 85 wt %, about 80 wt % to about 100 wt %, about 80 wt % to about 99 wt %, about 80 wt % to about 97 wt %, about 80 wt % to about 95 wt %, about 80 wt % to about 90 wt %, about 80 wt % to about 85 wt %, about 90 wt % to about 100 wt %, about 90 wt % to about 99 wt %, about 90 wt % to about 97 wt %, or about 90 wt % to about 95 wt %.

Additionally, the feed may comprise paraffins in an amount of about 0.0 wt %, at least about 5.0 wt %, at least about 10 wt %, at least about 20 wt %, at least about 30 wt %, at least about 40 wt %, at least about 50 wt %, at least about 60 wt %, at least about 65 wt %, at least about 70 wt %, at least about 75 wt %, at least about 80 wt %, at least about 85 wt %, at least about 90 wt %, at least about 95 wt %, at least about 97 wt %, at least about 99 wt % or up to about 100 wt %. Additionally or alternatively, the feed may comprise paraffins in an amount of about 5.0 wt % to about 20 wt %, about 5.0 wt % to about 10 wt %, about 5.0 wt % to about 100 wt %, about 20 wt % to about 100 wt %, about 50 wt % to about 100 wt %, about 60 wt % to about 100 wt %, about 60 wt % to about 99 wt %, about 60 wt % to about 97 wt %, about 60 wt % to about 95 wt %, about 60 wt % to about 90 wt %, about 60 wt % to about 85 wt %, about 70 wt % to about 100 wt %, about 70 wt % to about 99 wt %, about 70 wt % to about 97 wt %, about 70 wt % to about 95 wt %, about 70 wt % to about 90 wt %, about 70 wt % to about 85 wt %, about 80 wt % to about 100 wt %, about 80 wt % to about 99 wt %, about 80 wt % to about 97 wt %, about 80 wt % to about 95 wt %, about 80 wt % to about 90 wt %, about 80 wt % to about 85 wt %, about 90 wt % to about 100 wt %, about 90 wt % to about 99 wt %, about 90 wt % to about 97 wt %, or about 90 wt % to about 95 wt %

In some instances where the feed comprises olefins in an amount less than 100 wt %, the balance of the feed may be paraffins. For example, the feed may comprise about 50 wt % to about 100 wt % olefins and about 0.0 wt % to about 50 wt % paraffins. Alternatively, the feed may comprise about 5.0 wt % to about 20 wt % olefins and about 80 wt % to about 95 wt % paraffins. In particular, the feed may comprise combinations of ethane, ethene, propane and propene.

The olefins in the feed may be obtained utilizing existing process streams within a hydrocarbon refining plant, from chemical grade olefin sources, or a mixture thereof. In one embodiment, the olefins may be obtained from fuel gas, chemical grade propylene, refinery grade propylene, polymer grade propylene, liquefied petroleum gas (LPG), light cracked naphtha (LCN) process streams, scanfinate (hydroprocessed LCN) process streams, de-hydrogenated INN process streams (light virgin naphtha), and butylene or butylene-containing process streams (e.g., an alkylation feed). In another embodiment, the olefin feed composition may be obtained from a fluid catalytic cracking (FCC) coking operation, such as a FCC off-gas or coker off-gas stream, or from a steam cracking operation. In another embodiment, the olefins may be obtained from natural gas and coal sources via conversion of methanol and other oxygenates with the use of zeolite catalysts.

### II.B. Process Conditions

As previously discussed, the olefins may be oligomerized to produce the distillate boiling range product at advantageously lower pressures. In various embodiments, the reactor may be operated at a pressure of below about 600 psig, below or equal to about 500 psig, below or equal to about 400 psig, below or equal to about 300 psig, below or equal to about 200 psig, below or equal to about 100 psig, below or equal to about 90 psig or about 50 psig . In particular, the reactor may be operated at a pressure of below about 200 psig or below about 100 psig. Additionally or alternatively, the reactor may be operated at a pressure of about 50 psig to about 600 psig, about 90 psig to about 600 psig, about 90 psig to about 500 psig, about 90 psig to about 400 psig, about 90 psig to about 300 psig, about 90 psig to about 200 psig, about 90 psig to about 100 psig, about 100 psig to about 600 psig, about 100 psig to about 500 psig, about 100 psig to about 400 psig, about 100 psig to about 300 psig or about 100 psig to about 200 psig. In particular, the reactor may be operated at a pressure of about 90 psig to about 300 psig or about 90 psig to about 200 psig.

Additionally, the reactor may be operated at a suitable temperature for oligomerizing the olefins present in the feed. For example, in combination with the above-described pressures, the reactor may be operated at a temperature of about 100° C. to about 500° C., about 100° C. to about 400° C., about 100° C. to about 300° C., about 150° C. to about 300° C., about 150° C. to about 250° C., or about 200° C. to about 250° C. In particular, the reactor may be operated at a temperature of about 150° C. to about 300° C.

Further, the process conditions may include a mass of feed per mass of catalyst per hour (WHSV) of about 0.1 to about 20.

In various embodiments, the reactor may be a fixed bed (or packed bed) or a fluid bed reactor.

### II.C. Oligomerization Catalysts

A suitable oligomerization catalyst may be contained in the reactor. The oligomerization catalyst may comprise a molecular sieve material, such as a zeolite having 10-membered or 12-membered rings, particularly, 10-membered rings. In various aspects, the oligomerization catalyst may comprise a molecular sieve material having a framework structure selected from the following group of framework structures: BEA, FER, MEL, MFI, MRE, MFS, MTT, MTW, MWW, MOR, MEI, ITN, TON, and combinations thereof. In particular, the oligomerization catalyst may comprise a zeolite having a framework structure of MRE.

Suitable zeolites can include, but are not necessarily limited to, ZSM-5, ZSM-11, ZSM-12, ZSM-18, ZSM-22, ZSM-23, ZSM-35 ZSM-48, ZSM-57, MCM-49, MCM-22, ITQ-39 and the like, as well as intergrowths and combinations thereof. In certain embodiments, the zeolite can comprise, consist essentially of, or be ZSM-48.

Additionally or alternatively, the zeolite may be present at least partly in hydrogen form in the catalyst (e.g., HZSM-5, HZSM-48). Depending on the conditions used to synthesize the zeolite, this may implicate converting the zeolite from, for example, the alkali (e.g., sodium) form. This can readily be achieved, e.g., by ion exchange to convert the zeolite to the ammonium form, followed by calcination in air or an inert atmosphere at a temperature from about 400° C. to about 700° C. to convert the ammonium form to the active hydrogen form. If an organic structure directing agent is used in the synthesis of the zeolite, additional calcination may be desirable to remove the organic structure directing agent.

A person of ordinary skill in the art knows how to make the aforementioned frameworks and molecular sieves. For example, see the references provided in the International Zeolite Association's database of zeolite structures found at www.iza-structure.org/databases.

In further embodiments, the oligomerization catalyst may have a silicon to aluminum molar ratio of about 20 to about 120, about 20 to about 100, about 20 to about 90, about 20 to about 75, about 20 to about 60, about 20 to about 50, about 25 to about 45 or about 20 to about 30. In particular, the oligomerization catalyst may have a silicon to aluminum molar ratio of about 20 to about 100, about 25 to 45 or about 20 to about 30.

The surface area of the oligomerization catalyst can be determined, for example, using nitrogen adsorption-desorption isotherm techniques within the expertise of one of skill in the art, such as the BET (Brunauer Emmet Teller) method. As used herein, and unless otherwise specified, “surface area” refers to the microporous surface area as determined by the BET method.

In various embodiments, the oligomerization catalyst may have a surface area of greater than or equal to about 125 m²/g, greater than or equal to about 150 m²/g, greater than or equal to about 175 m²/g, greater than or equal to about 200 m²/g, greater than or equal to about 225 m²/g, greater than or equal to about 250 m²/g, greater than or equal to about 275 m²/g, greater than or equal to about 300 m²/g, greater than or equal to about 350 m²/g, greater than or equal to about 400 m²/g, greater than or equal to about 450 m²/g or about 500 m²/g. In particular, the oligomerization catalyst may have a surface area of greater than about 150 m²/g. Additionally or alternatively, the oligomerization catalyst may have a surface area of about 125 m²/g to about 500 m²/g, about 150 m²/g to about 500 m²/g, about 150 m²/g to about 300 m²/g, about 150 m²/g to about 250 m²/g, about 150 m²/g to about 225 m²/g, or about 150 m²/g to about 200 m²/g.

Additionally or alternatively, the oligomerization catalyst may have a hexane cracking activity of greater than or equal to about 10, greater than or equal to about 20, greater than or equal to about 40, greater than or equal to about 60, greater than or equal to about 80, greater than or equal to about 100, greater than or equal to about 120, greater than or equal to about 140, greater than or equal to about 160, greater than or equal to about 180, or greater than or equal to about 200, greater than or equal to about 400, greater than or equal to about 600, greater than or equal to about 800, greater than or equal to about 1000, greater than or equal to about 1200, greater than or equal to about 1400, or about 1500. In particular, the oligomerization catalyst may have a hexane cracking activity of greater than or equal to about 20. Additionally or alternatively, the oligomerization catalyst may have a hexane cracking activity of about 20 to about 1500, about 40 to about 1200, about 60 to about 1000, about 80 to about 600, about 100 to about 400, about 100 to about 200, about 100 to about 180. Hexane cracking activity as discussed herein may be determined according to U.S. Pat. No. 3,354,078; (1965) J. Catal., 4:527; (1966) J. Catal., 6:278; and (1980) J. Catal., 61:395.

In one embodiment, the oligomerization catalyst may have one or more of: a silicon to aluminum molar ratio as described herein; a surface area described herein; and a hexane cracking activity as described herein. In particular, the oligomerization catalyst may have one or more of: (i) a silicon to aluminum molar ratio of about 20 to about 100 or about 25 to about 45; (ii) a surface area greater than about 150 m²/g; and (iii) a hexane cracking activity of greater than about 20. Additionally or alternatively, the oligomerization catalyst may have two of (i), (ii) and (iii), e.g., (i) and (ii), (i) and (iii), or (ii) and (iii). Additionally or alternatively, the oligomerization catalyst may have three of (i), (ii) and (iii).

In the processes described herein and even at the lower pressures described herein, the oligomerization catalysts can convert at least about 50 wt % of olefins present in the feed (based on total weight of the feed) to oligomerized olefins to produce the distillate boiling range product. Further, the oligomerization catalysts can convert at least about 60 wt % of olefins, at least about 70 wt % of olefins, at least about 80 wt % of olefins, at least about 90 wt % of olefins, at least about 95 wt % of olefins, at least about 99 wt %, or about 100% of olefins present in the feed to oligomerized olefins to produce the distillate boiling range product. Additionally or alternatively, the oligomerization catalysts can convert about 50 wt % to about 100 wt % of olefins, about 60 wt % to about 100 wt % of olefins, about 70 wt % to about 100 wt % of olefins, about 80 wt % to about 100 wt % of olefins, about 90 wt % to about 100 wt % of olefins, about 95 wt % to about 100 wt % about 50 wt % to about 99 wt % of olefins, about 60 wt % to about 99 wt % of olefins, about 70 wt % to about 99 wt % of olefins, about 80 wt % to about 99 wt % of olefins, about 90 wt % to about 99 wt % of olefins, or about 95 wt % to about 99 wt % of olefins present in the feed.

In a particular embodiment, a process for oligomerizing olefins to produce a distillate boiling range product is provided. The process may comprise contacting a feed consisting essentially of C₂-C₄ olefins with an oligomerization catalyst comprising a zeolite having a framework structure selected from the group consisting of BEA, FER, MRE, MFI, MEL, MWW, MTT, MTW, MFS, MOR, MEI, ITN, TON, and a combination thereof in at least one reactor operating under suitable conditions as described herein, particularly at lower pressures, to oligomerize at least a portion of the olefins to produce an effluent comprising the distillate boiling range product, wherein the at least one reactor operates at a pressure below 200 psig.

In a further embodiment, another process for oligomerizing olefins to produce a distillate boiling range product is provided. The process may comprise contacting a feed comprising olefins with an oligomerization catalyst comprising a zeolite having a framework structure of MRE in at least one reactor operating under suitable conditions as described herein to oligomerize at least a portion of the olefins to produce an effluent comprising the distillate boiling range product, wherein the at least one reactor operates at a pressure below 200 psig.

In a further embodiment, another process for oligomerizing olefins to produce a distillate boiling range product is provided. The process may comprise contacting a feed comprising olefins with an oligomerization catalyst comprising a zeolite having a framework structure of MRE in at least one reactor operating under suitable conditions as described herein to oligomerize at least a portion of the olefins to produce an effluent comprising the distillate boiling range product, wherein the at least one reactor operates at a pressure below 200 psig.

In another embodiment, a process for oligomerizing olefins to produce a distillate boiling range product is provided. The process may comprise contacting a feed comprising olefins with an oligomerization catalyst in at least one reactor operating under suitable conditions as described herein to oligomerize at least a portion of the olefins to produce an effluent comprising the distillate boiling range product; wherein the at least one reactor operates at a pressure below 200 psig; and wherein the oligomerization catalyst comprises a zeolite having a framework structure selected from the group consisting of BEA, FER, MRE, MFI, MEL, MWW, MTT, MTW, MFS, MOR, MEI, ITN, TON, and a combination thereof; and the oligomerization catalyst has the following:
(i) a silicon to aluminum molar ratio of about 20 to about 100;
(ii) a surface area greater than about 150 m²/g; and
(iii) a hexane cracking activity of greater than about 20.

### II.D. Effluent

As described herein, the process produces an effluent comprising the distillate boiling range product. The distillate boiling range product may be present in the effluent in an amount (based on the total weight of the effluent) or a yield of at least about 40 wt %, at least about 50 wt %, at least about 60 wt %, at least about 70 wt %, at least about 80 wt %, at least about 90 wt % or about 95 wt %. Additionally or alternatively, the distillate boiling range product may be present in the effluent in an amount or a yield of about 40 wt % to about 95 wt %, about 50 wt % to about 95 wt %, about 60 wt % to about 95 wt %, about 70 wt % to about 95 wt %, about 80 wt % to about 95 wt % or about 90 wt % to about 95 wt %.

Additionally or alternatively, the effluent may comprise naphtha boiling range components in an amount (based on the total weight of the effluent) of at least about 1.0 wt %, at least about 5.0 wt %, at least about 10 wt %, at least about 20 wt %, at least about 30 wt %, or about 40 wt %. Further, the effluent may comprise naphtha boiling range components in an amount of about 1.0 wt % to about 40 wt %, about 1.0 wt % to about 30 wt %, about 1.0 wt % to about 20 wt % or about 5.0 wt % to about 10 wt %.

Additionally or alternatively, the effluent may comprise hydrocarbon components boiling above about 730° F. in an amount (based on the total weight of the effluent) of at least about 1.0 wt %, at least about 5.0 wt %, at least about 10 wt %, at least about 15 wt or about 2 wt %. Further, the effluent may comprise hydrocarbon components boiling above about 730° F. in an amount of about 1.0 wt % to about 20 wt %, about 1.0 wt % to about 15 wt %, about 1.0 wt % to about 10 wt % or about 1.0 wt % to about 5.0 wt %.

### II.E. Optional Steps

As discussed herein, the olefins in the feed may be obtained from existing process streams within a hydrocarbon refining plant, from chemical grade olefin sources, or a mixture thereof. For example, the olefins may be obtained from natural gas and coal sources via conversion of methanol and other oxygenates with the use of zeolite catalysts.

Thus, in certain variations, the processes described herein may further comprise contacting a first stream comprising methanol and/or dimethyl ether with a methanol conversion catalyst under suitable conditions as well-known in the art to produce the feed comprising olefins.

In a particular embodiment, a process for producing a distillate boiling range product is provided. The process may comprise contacting a first stream comprising methanol and/or dimethyl ether with a methanol conversion catalyst under suitable conditions to produce an intermediate product comprising olefins; and contacting the intermediate product with an oligomerization catalyst comprising a zeolite having a framework structure selected from the group consisting of BEA, FER, MRE, MEI, MEL, MWW, MTT, MTW, MES, MOR, MEI, ITN, TON, and a combination thereof at a pressure below 200 psig to oligomerize at least a portion of the olefins to produce the distillate boiling range product. Such a process comprises a first conversion to olefins step and a second olefin oligomerization step. The second olefin oligomerization step may be performed with the catalysts described herein and under the conditions described herein. The first and second step may be performed in the same or different reactor as described herein (e.g., fixed bed, fluid bed).

During the first conversion to olefins step, the methanol conversion catalyst may convert at least about 50 wt %, at least about 60 wt %, at least about 70 wt %, at least about 80 wt %, at least about 90 wt % or about 95 wt % of the methanol and/or dimethyl ether in the first stream to olefins. Additionally or alternatively, the methanol conversion catalyst may convert about 50 wt % to about 95 wt %, about 60 wt % to about 95 wt, about 70 wt % to about 95 wt %, about 80 wt % to about 95 wt %, or about 90 wt % to about 95 wt % of the methanol and/or dimethyl ether in the first stream to olefins.

The first stream may contact the methanol conversion catalyst at suitable conditions known in the art to convert the methanol and/or dimethyl ether to olefins. For example, such conditions may include a pressure of about 10 psig to about 100 psig or about 15 psig to about 75 psig and temperature of about 250° C. to about 600° C., about 300° C. to about 500° C., or about 300° C. to about 400° C.

Additionally, the methanol conversion catalyst may comprise a zeolite having a framework structure selected from the group consisting of BEA, FER MEI, MEL, MWW, MSE, MTW, MOR, MEI, ITN, TON, and a combination thereof.

Suitable zeolites can include, but are not necessarily limited to, ZSM-5, ZSM-11, ZSM-12, ZSM-18, ZSM-22, ZSM-35, MCM-68, MCM-49, MCM-22, ITQ-39, and the like, as well as intergrowths and combinations thereof. Additionally or alternatively, the zeolite may be present at least partly in hydrogen form in the catalyst (e.g., HZSM-5) as described herein.

### III. Distillate Boiling Range Product

A distillate boiling range product is also provided, particularly, where the distillate boiling range product is made according to the processes described herein. The distillate boiling range product may have a low aromatics and/or sulfur content. For example, the distillate boiling range product may have an aromatics content of less than about 10 vol %, less than about 8.0 vol %, less than about 5.0 vol %, less than about 3.0 vol %, less than about 1.0 vol % or about 0.10 vol %. Additionally or alternatively, the distillate boiling range product may have an aromatics content of about 0.10 vol % to about 10 vol %, about 1.0 vol % to about 10 vol %, or about 3.0 vol % to about 8.0 vol %.

Additionally or alternatively, the distillate boiling range product may have a sulfur content of less than about 2.0 wt %, less than about 1.0 wt %, less than about 0.10 wt %, less than about 0.010 wt %, less than about 0.0050 wt %, less than about 0.0020 wt %, less than about 0.0010 wt % or about about 0.00010 wt %. Additionally or alternatively, the distillate boiling range product may have a sulfur content of about 0.00010 wt % to about 2.0 wt %, about 0.00010 wt % to about 1.0 wt %, or about 0.0010 wt % to about 0.010 wt %.

In particular, the distillate boiling range product may have an aromatics content of less than about 5.0 vol % and/or a sulfur content of less than about 0.0020 wt %.

### IV. Reaction System for Oligomerizing Olefins

Reaction systems for oligomerizing olefins to produce a distillate boiling range product are also provided herein. The reaction system may comprise a feed stream comprising olefins as described herein, an effluent stream comprising the distillate boiling range product as described herein, and at least one reactor as described herein operated under suitable conditions as described herein to oligomerize at least a portion of the olefins to the distillate boiling range product. The feed may comprise, consist essentially of, or consist of C₂-C₅ olefins or C₂C₄ olefins in the amounts as described herein.

The at least one reactor may comprise a feed stream inlet for providing the feed stream to the reaction system, an oligomerization catalyst as described herein and an effluent outlet for removal of the effluent stream. Exemplary oligomerization catalysts include, but are not limited to, a zeolite having a framework structure selected from the group consisting of BEA, FER, MRE, MFI, MEL, MWW, MTT, MTW, MFS, MOR, MEI, ITN, TON, and a combination thereof. Suitable zeolites can include, but are not necessarily limited to, ZSM-5, ZSM-11, ZSM-12, ZSM-18, ZSM-22, ZSM-23, ZSM-35, ZSM-48, ZSM-57, MCM-49, MCM-22, ITQ-39, and the like, as well as intergrowths and combinations thereof as well as the hydrogen form of the zeolite. Additionally or alternatively, the oligomerization catalyst may have one or more of: (i) a silicon to aluminum molar ratio of about 20 to about 100 or about 25 to about 45; (ii) a surface area greater than about 150 m²/g; and (iii) a hexane cracking activity of greater than about 20. The at least one reactor may operate under the conditions described herein, particularly at lower pressures, for oligomerizing olefins to produce the distillate boiling range product, e.g., at a pressure below 200 psig or below 100 psig and a temperature of about 150° C. to about 300° C.

### V. Further Embodiments

The invention can additionally or alternatively include one or more of the following embodiments.

Embodiment 1. A process for oligomerizing olefins to produce a distillate boiling range product, wherein the process comprises contacting a feed consisting essentially of C₂-C₄ olefins with an oligomerization catalyst comprising a zeolite having a framework structure selected from the group consisting of BEA, FER, MRE, MFI, MEL, MWW, MTT, MTW, MFS, MOR, MEI, ITN, TON, and a combination thereof (e.g., ZSM-5, ZSM-11, MCM-22, ZSM-35, ZSM-48, ZSM-57, ZSM-12, MCM-49, ZSM-23, ZSM-18, ZSM-22, ITQ-39, and combinations thereof) in at least one reactor (e.g., fixed bed, fluid bed) operating under suitable conditions (e.g., a temperature of about 150° C. to about 300° C.) to oligomerize at least a portion of the olefins to produce an effluent comprising the distillate boiling range product, wherein the at least one reactor operates at a pressure below 200 psig, preferably below about 100 psig and optionally, wherein the oligomerization catalyst has one or more of the following:
(i) a silicon to aluminum molar ratio of about 20 to about 100, preferably about 25 to about 45;
(ii) a surface area greater than about 150 m²/g; and
(iii) a hexane cracking activity of greater than about 20.

Embodiment 2. A process for oligomerizing olefins to produce a distillate boiling range product, wherein the process comprises contacting a feed comprising olefins (e.g., C₂-C₅ olefins) with an oligomerization catalyst comprising a zeolite having a framework structure of MRE (e.g., ZSM-48 and/or H-ZSM-48) in at least one reactor (e.g., fixed bed, fluid bed) operating under suitable conditions (e.g., a temperature of about 150° C. to about 300° C.) to oligomerize at least a portion of the olefins to produce an effluent comprising the distillate boiling range product, wherein the at least one reactor operates at a pressure below 200 psig, preferably below about 100 psig and optionally, wherein the oligomerization catalyst has one or more of the following:
(i) a silicon to aluminum molar ratio of about 20 to about 100, preferably about 25 to about 45;
(ii) a surface area greater than about 150 m²/g; and
(iii) a hexane cracking activity of greater than about 20.

Embodiment 3. A process for oligomerizing olefins to produce a distillate boiling range product, wherein the process comprises contacting a feed comprising olefins (e.g., C₂-C₅ olefins, C₂-C₄ olefins) with an oligomerization catalyst in at least one reactor (e.g., fixed bed, fluid bed) operating under suitable conditions (e.g., a temperature of about 150° C. to about 300° C.) to oligomerize at least a portion of the olefins to produce an effluent comprising the distillate boiling range product; wherein the at least one reactor operates at a pressure below 200 psig, preferably below about 100 psig; and wherein the oligomerization catalyst comprises a zeolite having a framework structure selected from the group consisting of BEA, FER, MRE, MFI, MEL, MWW, MTT, MTW, MFS, MOR, MEI, ITN, TON, and a combination thereof (e.g., ZSM-5, ZSM-11, MCM-22, ZSM-35, ZSM-48, ZSM-57, ZSM-12, MCM-49, ZSM-23, ZSM-18, ZSM-22, ITQ-39, and combinations thereof); and the oligomerization catalyst has the following:
(i) a silicon to aluminum molar ratio of about 20 to about 100, preferably about 25 to about 45;
(ii) a surface area greater than about 150 m²/g; and
(iii) a hexane cracking activity of greater than about 20.

Embodiment 4. The process of any one of embodiments 1-3, wherein the oligomerization catalyst converts at least about 70 wt % of the olefins in the feed.

Embodiment 5. The process of any one of embodiments 1-4, wherein the effluent comprises at least about 50 wt % of the distillate boiling range product.

Embodiment 6. The process of any one of embodiments 1-5 further comprising contacting a first stream comprising methanol and/or dimethyl ether with a methanol conversion catalyst under suitable conditions to produce the feed.

Embodiment 7. A process for producing a distillate boiling range product, wherein the process comprises: contacting a first stream comprising methanol and/or dimethyl ether with a methanol conversion catalyst (e.g., comprising a zeolite having a framework structure selected from the group consisting of MFI, MEL, MSE, MTW and a combination thereof) under suitable conditions (e.g., a temperature of about 300° C. to about 500° C. and a pressure of about 15 psig to about 75 psig) to produce an intermediate product comprising olefins (e.g., comprising C₂-C₅ olefins, consisting essentially of C₂-C₄ olefins); and contacting the intermediate product with an oligomerization catalyst comprising a zeolite having a framework structure selected from the group consisting of BEA, FER, MRE, MFI, MEL, MWW, MTT, MTW, MFS, and a combination thereof (e.g., ZSM-5, ZSM-11, ZSM-35, MCM-22, ZSM-48, ZSM-57, ZSM-12, MCM-49, ZSM-23 and a combination thereof) at a pressure below 200 psig, preferably below about 100 psig, to oligomerize at least a portion of the olefins to produce the distillate boiling range product and optionally, wherein the oligomerization catalyst has one or more of the following:
(i) a silicon to aluminum molar ratio of about 20 to about 100, preferably about 25 to about 45;
(ii) a surface area greater than about 150 m²/g; and
(iii) a hexane cracking activity of greater than about 20.

Embodiment 8. The process of embodiment 7, wherein the methanol conversion catalyst converts from about 90% to about 95% of the methanol and/or dimethyl ether in the first stream.

Embodiment 9. The process of embodiment 7 or 8, wherein the intermediate product contacts the oligomerization catalyst at a temperature of about 150° C. to about 300° C.

Embodiment 10. The process of any one of embodiments 7-9, wherein the oligomerization catalyst converts at least about 90 wt % of the olefins in the intermediate product.

Embodiment 11. The process of any one of embodiments 7-10, wherein the effluent comprises at least about 50 wt % of the distillate boiling range product.

Embodiment 12. The process of any one of embodiments 7-11, wherein the first stream contacts the methanol conversion catalyst and the intermediate product contacts the oligomerization catalyst in a same or different reactor (e.g., a fixed bed or a fluid bed).

Embodiment 13. A distillate boiling range product made according to the process of any one of the previous embodiments, wherein the distillate boiling range product has an aromatics content of less than about 5.0 vol % and/or a sulfur content of less than about 0.0020 wt %.

Embodiment 14. A reaction system for oligomerizing olefins to produce a distillate boiling range product comprising: a feed stream comprising olefins (e.g., comprising C₂-C₅ olefins, consisting essentially of C₂-C₄ olefins); an effluent stream comprising the distillate boiling range product; and at least one reactor (e.g., fixed bed, fluid bed) operated under suitable conditions (e.g., a temperature of about 150° C. to about 300° C.) to oligomerize at least a portion of the olefins to the distillate boiling range product, wherein the at least one reactor comprises: a feed stream inlet for providing the feed stream to the reaction system; a catalyst comprising a zeolite having a framework structure selected from the group consisting of BEA, FER, MRE, MFI, MEL, MWW, MTT, MTW, MFS, MOR, MEI, ITN, TON, and a combination thereof (e.g., ZSM-5, ZSM-11, MCM-22, ZSM-48, ZSM-35, ZSM-57, ZSM-12, MCM-49, ZSM-23, ZSM-18, ZSM-22, ITQ-39 and combinations thereof); and an effluent outlet for removal of the effluent stream; and wherein the at least one reactor operates at a pressure below 200 psig, preferably below about 100 psig and optionally, wherein the oligomerization catalyst has one or more of the following:
(i) a silicon to aluminum molar ratio of about 20 to about 100, preferably about 25 to about 45;
(ii) a surface area greater than about 150 m²/g; and
(iii) a hexane cracking activity of greater than about 20.

Embodiment 15. The reaction system of embodiment 14, wherein the oligomerization catalyst converts at least about 90 wt % of the olefins in the feed stream.

Embodiment 16. The reaction system of embodiment 14 or 15, wherein the effluent stream comprises at least about 50 wt % of the distillate boiling range product.

### EXAMPLES

### General Catalyst Information

Details regarding the catalysts used in the examples are provided below in Table 1.

TABLE 1

| | | | Total BET | Micropore |
|---|---|---|---|---|
| | | | Surface | Surface |
| Catalyst | SiO₂/Al₂O₃ | Alpha | Area (m²/g) | Area(m²/g) |
| H-ZSM-5 | 62 | 440 | 487 | 409 |
| H-ZSM-23 | 122 | 47 | 356 | 248 |
| H-ZSM-48 | 76 | 130 | 333 | 172 |
| H-MCM-22 | 42 | 490 | 575 | 496 |
| H-ZSM-12 | 181 | 57 | 391 | 314 |
| H-ZSM-57 | 40 | 1200 | 480 | 434 |
| MCM-49 | 18 | 680 | 565 | 463 |

### Example 1

### Conversion of Light Olefins to Distillate Boiling Range Product with H-ZSM-48

### Example 1a

### Experiment Performed at 200° C. and Varying Pressures

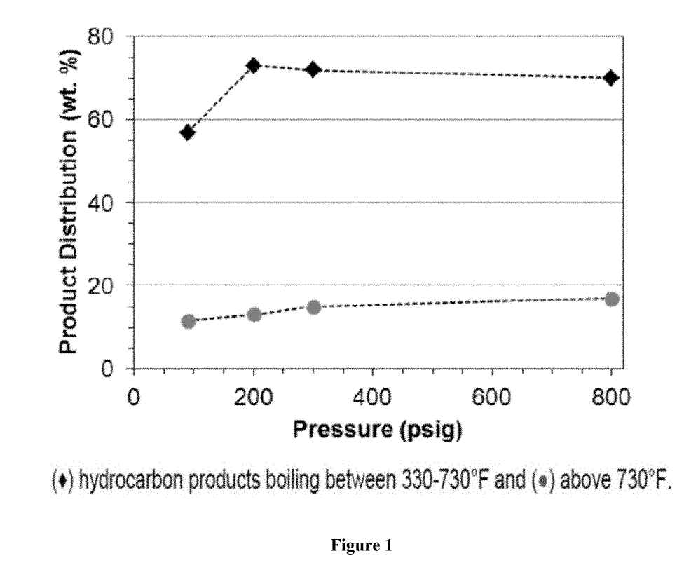

A propene stream was passed over an H-ZSM-48 catalyst at a temperature of 200° C. at a mass of feed per mass of catalyst per hour (WHSV) of 1.67 in a fixed bed under steady state at different pressures to oligomerize propene and form a distillate boiling range product. The H-ZSM-48 catalyst had a silicon to aluminum ratio of 76, a microporous surface area of 172 m²/g and a hexane cracking activity of 130. As is shown in FIG. 1, a high yield of distillate boiling range product (55 to 75 wt % of total hydrocarbons boiling between 330° C. and 730° C.) was produced from propene. Between 200 and 800 psig, the product yield was independent of pressure. While such yields are common for other catalysts at pressures above 600 psig, surprisingly, H-ZSM-48 was able to maintain yields above 50 wt % at pressures as low as 90 psig.

### Example 1b

### Experiment Performed at 225° C. and a Pressure of 200 psig and 800 psig

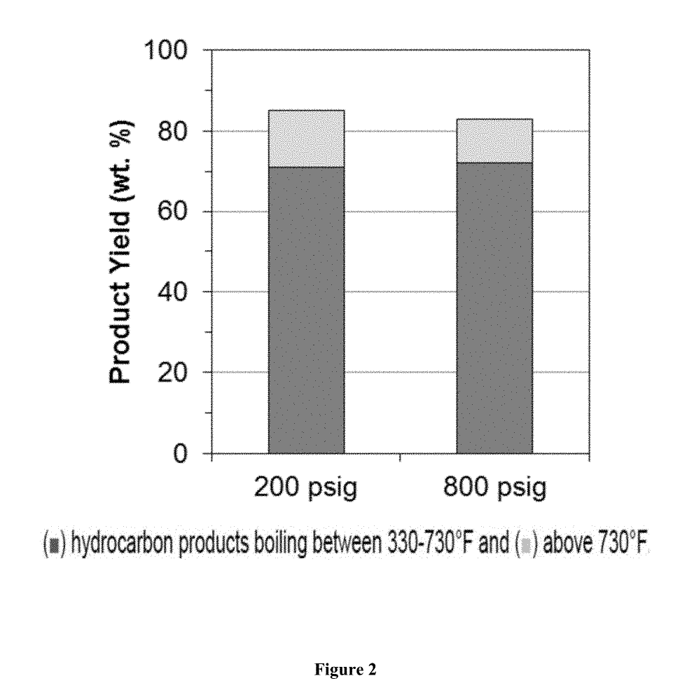

Using the same catalyst, H-ZSM-48, a 1-pentene stream was passed over the H-ZSM-48 catalyst at a temperature of 225° C. and WHSV of 1.67 at 200 psig and 800 psig in a fixed bed under steady state to oligomerize propene and form a distillate boiling range product. As shown in FIG. 2, the yield to distillate boiling range product was relatively unchanged (˜72 wt %) at the tested conditions despite the 600 psi difference in pressure.

The data in FIGS. 1 and 2 indicates that H-ZSM-48 can catalyze the transformation of LPG-range and naphtha-range olefins into more valuable distillate boiling range olefins. Likewise, H-ZSM-48 was able to convert these light olefins to distillate with yields greater than 70 wt % at lower pressure than what is currently preferred (600 psig or greater) for light olefin conversion to distillate.

### Example 2

### Comparison of Catalysts for Conversion of Light Olefins to Distillate Boiling Range Product

### Example 2a

### Experiment Performed at 200° C. and a Pressure of 800 psig and WHSV of 1.67

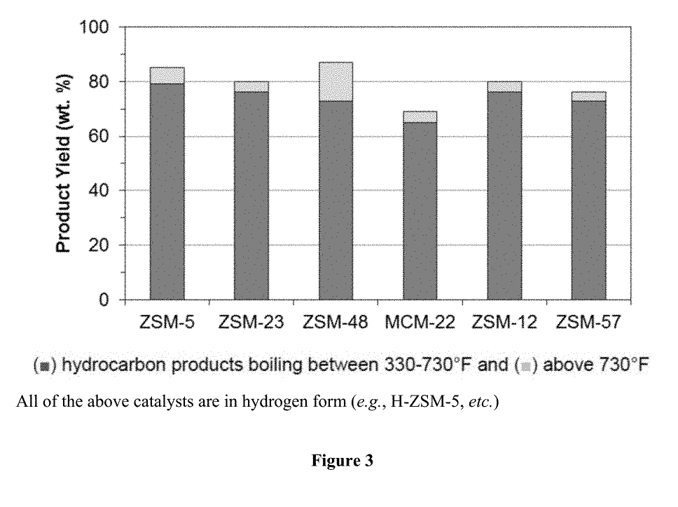

A propene stream was passed over six catalysts with different zeolite frameworks at a temperature of 200° C. and WHSV of 1.67 at 800 psig in a fixed bed under steady state to oligomerize propene and form a distillate boiling range product. The six catalysts tested were H-ZSM-5, H-ZSM-23, H-ZSM-48, H-MCM-22, H-ZSM-12, and H-ZSM-57. For all catalysts tested, the liquid product accounted for 98-99% of the total reactor effluent. As shown in FIG. 3, H-ZSM-48 produced the highest yield of hydrocarbons with boiling points 330° F. and above with a yield of 87 wt %. On the other hand, the yield to distillate boiling range product (boiling between 330° F. and 730° F.) was highest for H-ZSM-5 with a yield of 79 wt %. These results show that at conventional pressures (>600 psig) preferred for olefin conversion to distillate boiling range product, H-ZSM-5 is the preferred catalyst. On the other hand, H-ZSM-48 was able to maintain high yields of distillate boiling range product at low pressure.

### Example 2b

### Experiment Performed at 200° C., a Pressure of 90 psig and WHSV of 1.67

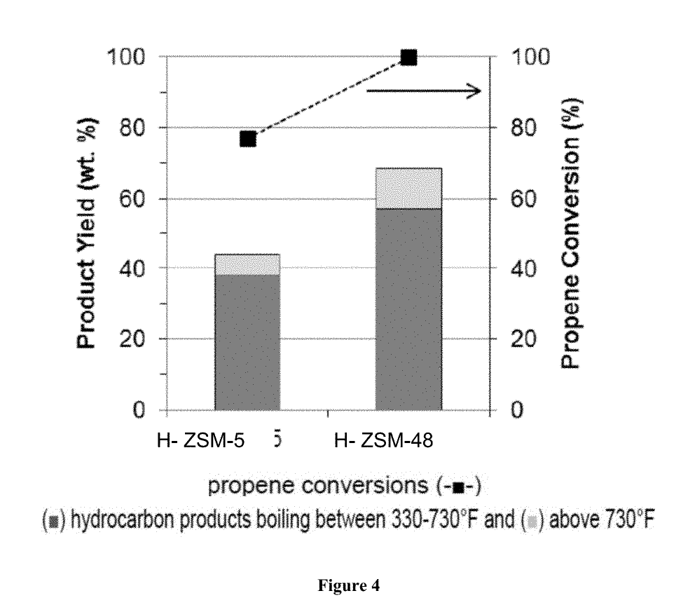

A propene stream was passed over H-ZSM-5 and H-ZSM-48 catalysts at a temperature of 200° C. and WHSV of 1.67 at 90 psig in a fixed bed under steady state to oligomerize propene and form a distillate boiling range product. As shown in FIG. 4, distillate boiling range product yield is provided when using H-ZSM-48. Further the conversion of propene with H-ZSM-48 was >99% and the conversion using H-ZSM-5 was 77%.

### Example 2c

### Experiment Performed at 200° C., WHSV of 1.67 and Varying Pressures

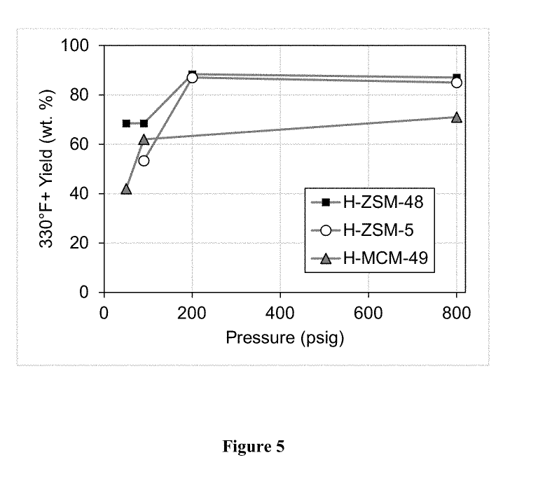

A propene stream was passed over H-ZSM-5, H-ZSM-48 and H-MCM-49 catalysts at a temperature of 200° C., WHSV of 1.67 in a fixed bed under steady state at different pressures (50-800 psig) to oligomerize propene and form a distillate boiling range product. The results are show in FIG. 5.

1. A process for oligomerizing olefins to produce a distillate boiling range product, wherein the process comprises contacting a feed consisting essentially of C₂-C₄ olefins with an oligomerization catalyst comprising a zeolite having a framework structure selected from the group consisting of BEA, FER, MRE, MEI, MEL, MWW, MTT, MTW, MES, MOR, MEI, ITN, TON, and a combination thereof in at least one reactor operating under suitable conditions to oligomerize at least a portion of the olefins to produce an effluent comprising the distillate boiling range product, wherein the at least one reactor operates at a pressure below 200 psig.

2. The process of claim 1, wherein the reactor operates at a pressure below about 100 psig.

3. The process of claim 1, wherein the reactor operates at a temperature of about 150° C. to about 300° C.

4. The process of claim 1, wherein the oligomerization catalyst converts at least about 70 wt % of the olefins in the feed.

5. The process of claim 1, wherein the effluent comprises at least about 50 wt % of the distillate boiling range product.

6. The process of claim 1, wherein the oligomerization catalyst has one or more of the following:
(i) a silicon to aluminum molar ratio of about 20 to about 100;
(ii) a surface area greater than about 150 m²/g; and
(iii) a hexane cracking activity of greater than about 20.

7. The process of claim 6, wherein the oligomerization catalyst has a silicon to aluminum molar ratio of about 25 to about 45.

8. The process of claim 1, wherein the oligomerization catalyst is selected from the group consisting of ZSM-5, ZSM-11, ZSM-35, MCM-22, ZSM-48, ZSM-57, ZSM-12, MCM-49, ZSM-23, ZSM-18, ZSM-22, ITQ-39 and a combination thereof

9. The process of claim 1, wherein the at least one reactor is a fixed bed or a fluid bed.

10. The process of claim 1, further comprising contacting a first stream comprising methanol and/or dimethyl ether with a methanol conversion catalyst under suitable conditions to produce the feed.

11. A process for oligomerizing olefins to produce a distillate boiling range product, wherein the process comprises contacting a feed comprising olefins with an oligomerization catalyst comprising a zeolite having a framework structure of MRE in at least one reactor operating under suitable conditions to oligomerize at least a portion of the olefins to produce an effluent comprising the distillate boiling range product, wherein the at least one reactor operates at a pressure below 200 psig.

12. The process of claim 11, wherein the reactor operates at a pressure below about 100 psig.

13. The process of claim 11, wherein the reactor operates at a temperature of about 150° C. to about 300° C.

14. The process of claim 11, wherein the feed comprises C₂-C₅ olefins.

15. The process of claim 11, wherein the oligomerization catalyst converts at least about 90 wt % of the olefins in the feed.

16. The process of claim 11, wherein the effluent comprises at least about 50 wt % of the distillate boiling range product.

17. The process of claim 11, wherein the oligomerization catalyst has one or more of the following:
(i) a silicon to aluminum molar ratio of about 20 to about 100;
(ii) a surface area greater than about 150 m²/g; and
(iii) a hexane cracking activity of greater than about 20.

18. The process of claim 17, wherein the oligomerization catalyst has a silicon to aluminum molar ratio of about 25 to about 45.

19. The process of claim 11, wherein the oligomerization catalyst is ZSM-48 and/or H-ZSM-48.

20. The process of claim 11, wherein the at least one reactor is a fixed bed or a fluid bed.

21. The process of claim 11, further comprising contacting a first stream comprising methanol and/or dimethyl ether with a methanol conversion catalyst under suitable conditions to produce the feed.

22. A process for oligomerizing olefins to produce a distillate boiling range product, wherein the process comprises contacting a feed comprising olefins with an oligomerization catalyst in at least one reactor operating under suitable conditions to oligomerize at least a portion of the olefins to produce an effluent comprising the distillate boiling range product;
wherein the at least one reactor operates at a pressure below 200 psig; and
wherein the oligomerization catalyst comprises a zeolite having a framework structure selected from the group consisting of BEA, FER, MRE, MFI, MEL, MWW, MTT, MTW, MFS, MOR, MEI, ITN, TON, and a combination thereof; and the oligomerization catalyst has the following:
(i) a silicon to aluminum molar ratio of about 20 to about 100;
(ii) a surface area greater than about 150 m²/g; and
(iii) a hexane cracking activity of greater than about 20.

23. A process for producing a distillate boiling range product, wherein the process comprises:
contacting a first stream comprising methanol and/or dimethyl ether with a methanol conversion catalyst under suitable conditions to produce an intermediate product comprising olefins; and
contacting the intermediate product with an oligomerization catalyst comprising a zeolite having a framework structure selected from the group consisting of BEA, FER, MRE, MFI, MEL, MWW, MTT, MTW, MFS, MOR, MEI, ITN, TON, and a combination thereof at a pressure below 200 psig to oligomerize at least a portion of the olefins to produce the distillate boiling range product.

24. The process of claim 23, wherein the methanol conversion catalyst converts from about 90% to about 95% of the methanol and/or dimethyl ether in the first stream.

25. The process of claim 23, wherein the methanol conversion catalyst comprises a zeolite having a framework structure selected from the group consisting of BEA, FER, MFI, MEL, MSE, MTW and a combination thereof

26. The process of claim 23, wherein the first stream contacts the methanol conversion catalyst at a temperature of about 300° C. to about 500° C. and a pressure of about 15 psig to about 75 psig.

27. The process of claim 23, wherein the intermediate product contacts the oligomerization catalyst at a pressure below about 100 psig.

28. The process of claim 23, wherein the intermediate product contacts the oligomerization catalyst at a temperature of about 150° C. to about 300° C.

29. The process of claim 23, wherein the intermediate product comprises C₂-C₅ olefins.

30. The process of claim 23, wherein the intermediate product consists essentially of C₂-C₄ olefins.

31. The process of claim 23, wherein the oligomerization catalyst converts at least about 90 wt % of the olefins in the intermediate product.

32. The process of claim 23, wherein the effluent comprises at least about 50 wt % of the distillate boiling range product.

33. The process of claim 23, wherein the oligomerization catalyst has one or more of the following:
(i) a silicon to aluminum molar ratio of about 20 to about 100;
(ii) a surface area greater than about 150 m²/g; and
(iii) a hexane cracking activity of greater than about 20.

34. The process of claim 33, wherein the oligomerization catalyst has a silicon to aluminum molar ratio of about 25 to about 45.

35. The process of claim 23, wherein the oligomerization catalyst is selected from the group consisting of ZSM-5, ZSM-11, ZSM-35, MCM-22, ZSM-48, ZSM-57, ZSM-12, MCM-49, ZSM-23, ZSM-18, ZSM-22, ITQ-39, and a combination thereof.

36. The process of claim 23, wherein the first stream contacts the methanol conversion catalyst and the intermediate product contacts the oligomerization catalyst in a same or different reactor.

37. The process of claim 36, wherein the reactor is a fixed bed or a fluid bed.

38. A distillate boiling range product made according to the process of claim 1, wherein the distillate boiling range product has an aromatics content of less than about 5.0 vol % and/or a sulfur content of less than about 0.0020 wt %.

39. A reaction system for oligomerizing olefins to produce a distillate boiling range product comprising:
a feed stream consisting essentially of C₂-C₄ olefins;
an effluent stream comprising the distillate boiling range product; and
at least one reactor operated under suitable conditions to oligomerize at least a portion of the olefins to the distillate boiling range product, wherein the at least one reactor comprises:
a feed stream inlet for providing the feed stream to the reaction system;
a catalyst comprising a zeolite having a framework structure selected from the group consisting of BEA, FER, MRE, MFI, MEL, MWW, MTT, MTW, MFS, MOR, MEI, ITN, TON, and a combination thereof; and
an effluent outlet for removal of the effluent stream; and
wherein the at least one reactor operates at a pressure below 200 psig.
